(19) United States
(12) Patent Application Publication
(10) Pub. No.: US 2014/0242657 A1
(43) Pub. Date: Aug. 28, 2014

(54) PROCESS FOR THE PRODUCTION OF ALCOHOLS AND/OR SOLVENTS FROM PAPERMAKING PULPS WITH RECYCLING OF NON-HYDROLYZED VEGETATION

(71) Applicants: Marcel ROPARS, Palaiseau (FR); Caroline AYMARD, Lyon (FR)

(72) Inventors: Marcel ROPARS, Palaiseau (FR); Caroline AYMARD, Lyon (FR)

(73) Assignee: IFP Energies nouvelles, Rueil-Malmaison Cedex (FR)

(21) Appl. No.: 14/269,486

(22) Filed: May 5, 2014

Related U.S. Application Data
(63) Continuation of application No. 13/518,523, filed on Oct. 5, 2012.

(30) Foreign Application Priority Data
Dec. 23, 2009 (FR) 09/06.335

(51) Int. Cl. C12P 7/14; C12P 7/16; C12P 7/28

(52) U.S. Cl. CPC C12P 7/14; C12P 7/28; C12P 7/16
USPC 435/150; 435/162; 435/160

(57) ABSTRACT

This invention describes a process for the production of alcohols and/or solvents from cellulosic or lignocellulosic biomass that comprises at least the following stages:
a) Alkaline chemical pretreatment based on sodium sulfate of a cellulosic or lignocellulosic substrate;
b) Washing of the pretreated substrate;
c) Enzymatic hydrolysis of the substrate that is pretreated and washed using cellulolytic and/or hemicellulolytic enzymes that produce a hydrolyzate and a water-insoluble residue;
d) Microorganism fermentation of the hydrolyzate that is obtained from stage c) and production of a fermentation must that contains at least one alcohol and/or solvent;
e) Separation/purification of alcohol and/or solvent, and
f) Separation of a cake that contains the insoluble residue, in which at least a portion of the cake that is obtained in stage f) is recycled upstream from the pretreatment stage a) and/or upstream from the washing stage b).

### FIELD OF THE INVENTION

This invention is part of the framework of a process for the production of so-called “second generation” alcohol and/or solvent from lignocellulosic biomass. It relates more particularly to a process for the production of ethanol and/or an acetone-butanol-ethanol mixture (also called an ABE mixture).

### PRIOR ART

The lignocellulosic biomass represents one of the most abundant renewable resources on earth. The substrates that are under consideration are very varied, since they relate both to ligneous substrates (leafy and resinous), the by-products of agriculture (straw), or those of the lignocellulosic waste-generating industries (farm produce and papermaking industries).

The lignocellulosic biomass consists of three primary polymers: cellulose (35 to 50%), hemicellulose (20 to 30%), which is a polysaccharide that consists essentially of pentoses and hexoses, and lignin (15 to 25%), which is a polymer of complex structure and high molecular weight, consisting of aromatic alcohols that are connected by ether bonds.

These different molecules are responsible for intrinsic properties of the vegetation wall and are organized in a complex intergrowth.

Cellulose and optionally hemicelluloses are the targets of enzymatic hydrolysis but are not directly accessible to the enzymes. This is the reason for which these substrates are to undergo a pretreatment preceding the enzymatic hydrolysis stage. The purpose of the pretreatment is to modify the physical and physico-chemical properties of the lignocellulosic material for the purpose of improving the accessibility of the cellulose that is imprisoned within the matrix of lignin and hemicellulose.

Numerous technologies for carrying out this pretreatment exist: acid baking, alkaline baking, vapor explosion, organosolv methods, etc. The effectiveness of pretreatment is measured both by the material balance at the end of the pretreatment (recovery level of sugars in soluble monomer or oligomer form or in insoluble polymer form) and also by the cellulosic and hemicellulosic residues' susceptibility to enzymatic hydrolysis.

The processes for the production of alcohols and/or solvents from lignocellulosic biomass, called “second-generation processes,” comprise at least the following stages:
Pretreatment of the substrate,
Enzymatic hydrolysis of the pretreated substrate,
Fermentation of the hydrolyzate that is obtained, and
Separation/purification of the alcohol and/or solvent that is obtained after fermentation.

The economic validity of this type of process for the production of alcohol and/or solvent is difficult to achieve even for the operators that have broad mobilizable resources. Two items have a strong impact on the overall expense: the enzymatic feedstock that is necessary for the hydrolysis of polymerized sugars and the pretreated vegetation material. The optimization of this type of process therefore requires optimum upgrading of the enzymatic feedstock that is expressed in terms of kg of sugars released per kg or FPu of added enzymes. These conditions are produced by means of low enzymatic feedstocks, typically 5 to 10 g/kg of dry material. Unfortunately, these slow enzymatic feedstocks do not enhance the pretreated substrate well because the hydrolysis yield is mediocre, in particular that of the glucans that constitute the essential target because the conversion of glucose into ethanol and ABE is easy.

The insoluble dry material that is subjected to enzymatic hydrolysis can vary by 5 to 40%, and in general between 10 and 25%. According to the publication by Kristensen et al., Biotechnology for Biofuels, 2009 (2) 11, for obtaining an identical hydrolysis yield, the enzymatic consumption is to be higher in the case of an elevated feedstock of insoluble dry material, in particular because of the deactivation of the enzyme by the products of enzymatic hydrolysis (glucose, cellobiose). The approach that would consist in carrying out dilution at the enzymatic hydrolysis stage is, however, limited since it will have significant consequences on the energy cost linked to the separation of the alcohol that is produced by distillation. In the specific case of the manufacturing of ethanol, an alcohol concentration of the fermentation must with 23-25 g/L of ethanol at a minimum (alcohol titre of 3) is necessary to ensure a reasonable cost for the distillation item.

In contrast, to optimize these processes, it is desirable to maximize the amount of hydrolyzable and enzyme-accessible raw material from an initial amount of biomass.

Beyond the improvements linked to the effectiveness of enzymatic cocktails, the improvement of the economic balance sheet of the production of ethanol or ABE can be obtained by means of recycling of different flows or products.

The Patent Application WO 94/29475 proposes an improved process for the conversion of cellulosic biomass into ethanol in which a portion of the effluents that are obtained from the fermenter is recycled at the inlet of the same fermenter as a source of nutrients for the microorganism that is used during the fermentation.

In the second-generation processes, to improve their economic profitability, an effort is made to maximize the amount of released sugars, and therefore to maximize the yield, while keeping consumption of products and enzymes the lowest possible.

This invention describes an improved process for the production of alcohols and/or solvents that is paired with a unit for the production of papermaking pastes, using an alkaline chemical pretreatment.

### SUMMARY OF THE INVENTION

This invention relates to a process for the production of so-called second-generation alcohols and/or solvents, in which the lignocellulosic or cellulosic biomass undergoes an alkaline pretreatment based on sodium sulfate, of the Kraft pretreatment type, in which intensive recycling of the pastes that are not enzymatically hydrolyzed is performed.

### DESCRIPTION OF THE DRAWINGS

FIG. 1 is a diagrammatic representation of a device that implements a process for the production of alcohols and/or solvents from papermaking pulps, comprising a stage for recycling solid residues, according to this invention.

### DETAILED DESCRIPTION OF THE INVENTION

This invention describes a process for the production of alcohols and/or solvents from cellulosic or lignocellulosic biomass that comprises at least the following stages:
a) Alkaline chemical pretreatment based on sodium sulfate of a cellulosic or lignocellulosic substrate;
b) Washing of the pretreated substrate;
c) Enzymatic hydrolysis of the substrate that is pretreated and washed using cellulolytic and/or hemicellulolytic enzymes that produce a hydrolyzate and a water-insoluble residue;
d) Microorganism fermentation of the hydrolyzate that is obtained from stage c) and production of a fermentation must that contains at least one alcohol and/or solvent;
e) Separation/purification of alcohol and/or solvent, and
f) Separation of a cake that contains the insoluble residue,
   in which at least a portion of the cake that is obtained in stage f) is recycled upstream from the pretreatment stage a) and/or upstream from the washing stage b).

Thus, owing to the process according to this invention, it is possible to improve the upgrading of the lignocellulosic substrate. Actually, the process makes it possible to use more than 80% by weight, and preferably more than 90% by weight, of the cellulose that is contained in the vegetation for its future alcohol conversion, and/or in an ABE mixture.

Owing to the recycling in the alkaline medium according to the invention, with or without heat treatment, the cellulose that is not hydrolyzed, also called recalcitrant cellulose, partially recovers its susceptibility to enzymatic hydrolysis. The term of recalcitrant cellulose, in the meaning of this invention, is defined as cellulose that is not hydrolyzed during stage c) for enzymatic hydrolysis and that has, without specific treatment, a mediocre susceptibility to enzymatic hydrolysis.

According to one embodiment of the invention, the recycling is carried out upstream from the pretreatment stage a). The thus applied alkaline treatment that is identical to the one carried out during the pretreatment stage a) makes possible the swelling of the fibers of the paste and regenerates the susceptibility of the thus pretreated substrate to enzymatic hydrolysis (mercerization), without giving rise to the accumulation of lignin.

Actually, by the recycling of a portion of the cake, considered as the indigestible fraction, upstream from the pretreatment stage, the lignin concentration is perfectly regulated and stabilized.

According to another embodiment of the invention, the recycling of at least one fraction of the cake is carried out upstream from the washing stage b): the non-hydrolyzed recalcitrant cellulose does not undergo heat treatment, and the energy that is dispensed is insignificant. The simple recycling in an alkaline medium before the washing stage makes it possible to considerably increase the susceptibility to enzymatic hydrolysis. This treatment is less effective, however, than the one described above.

According to another embodiment, the recycling of at least one fraction of the cake is carried out upstream from the pretreatment stage a) and upstream from the washing stage b).

The process according to this invention makes it possible to limit the amount of enzymes that are to be used for producing an overall hydrolysis of more than 90% of the cellulose of the initial pretreated substrate. Ultimately, the enzyme only encounters a substrate that is “very susceptible to enzymatic hydrolysis” and no longer encounters recalcitrant cellulose because of the recycling of the latter.

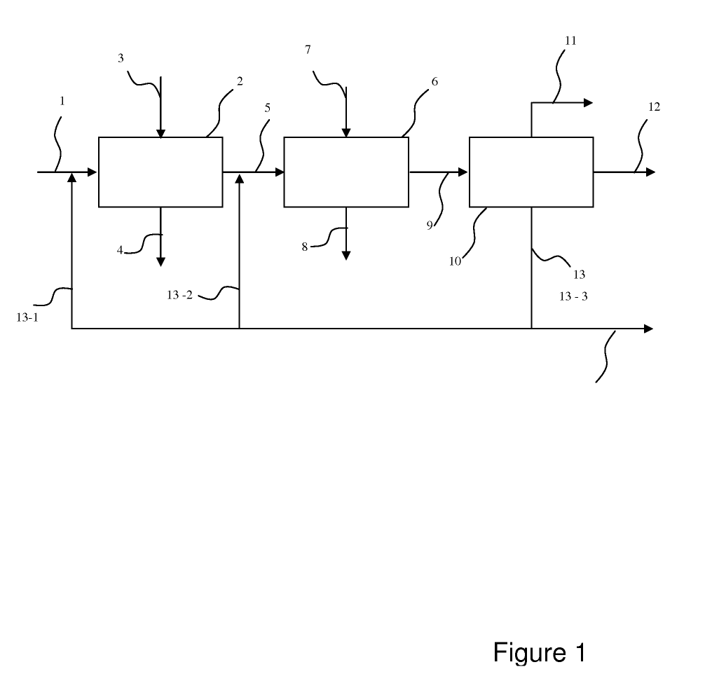

The invention will be described by referring to FIG. 1.

Stage a) for alkaline chemical pretreatment of the cellulosic or lignocellulosic substrate implements a process that is known under the name of sodium sulfate process or Kraft process based on the use of soda and sodium sulfate. It involves a process that is tested and validated economically since it is commonly used in papermaking processes.

The substrate that is used is selected from among the most varied biomasses, but more particularly from the resinous arborescent types (softwood such as spruce or pine) or leafy arborescent types (hardwood such as eucalyptus) or else agricultural lignocellulosic waste (corn straw, rice, etc.).

The biomass 1 is introduced into the cooking plant or else designated below as a digester 2. An alkaline solution that is based on sodium sulfate 3 is introduced into the digester 2.

The alkaline chemical treatment of the biomass is done at 150-180° C. for a period of 2 to 5 hours based on the substrate that is used. It is thus partially delignified by means of baking at high temperature and in the presence of soda. The baking is carried out in a vertical reactor, where the biomass chips drop by gravity and encounter the various baking liquors. Sodium sulfide is prepared directly from sodium sulfate by combustion. During baking, the sodium sulfide is hydrolyzed into soda, NaHS, and H₂S. The different sulfur-containing compounds that are present react with lignin to provide thiolignins that are more easily soluble.

In parallel with the baking stage, the Kraft process comprises a recycling loop of the chemical reagents that are used. The largest portion of the chemical products (soda and sodium sulfide) is recovered in the residual cooking liquors.

The lignin that is contained in the biomass is partially solubilized and is discharged with the spent alkaline solution 4, also called black liquor. This delignification is monitored by the operating parameters of the digesters. The black liquor 4 can contain hemicelluloses and very little cellulose.

At the end of the pretreatment stage a) according to the process of this invention, the pretreated substrate is obtained in the form of a cellulose-enriched paste 5 (also called “pulp”).

During the washing stage b) of the pretreated substrate, this paste 5 is washed in the reactor 6. One or more washing liquids 7 are introduced into said washing reactor 6. A more intensive delignification can be conducted during the washing stage that is carried out in the reactor 6. A separation tool such as a press or a centrifugal decanter can be installed for eliminating the alkalinity.

The spent washing liquid(s) 8 is/are removed at the outlet of the reactor 6.

The paste or washed pulp 9 that is extracted from the washing reactor 6 contains between 1% and 40% of solid material, preferably between 7% and 40%, and more preferably between 10% and 25%.

A neutralization of the paste can be conducted prior to the stage for enzymatic hydrolysis by the addition of acids. It is actually necessary that the enzymatic hydrolysis be carried out at a pH of between 4 and 5.5.

The pretreated and washed paste is next sent into the process for conversion of alcohols and/or solvents, shown diagrammatically by the rectangle 10, where the stages c) to e) are carried out, corresponding to the conversion stages themselves. These conversion stages can be two to eight in number. Preferably, there are between three and five of them.

These conversion stages comprise at least the stages c) and d) that correspond respectively to an enzymatic hydrolysis and a fermentation of the pulp. These stages can optionally be coupled in the same reactor. Reference is then made to the SSF (“Simultaneous Saccharification and Fermentation”) process.

The enzymatic hydrolysis stage c) is carried out by means of enzymes of the cellulases and/or hemicellulases type produced by a microorganism. In a preferred way, the microorganism that is used is a mushroom that belongs to the genera Trichoderma, Aspergillus, Penicillium or Schizophyllum, or an anaerobic bacteria that belongs to the genus Clostridium. In a very preferred way, the microorganism that is used is Trichoderma reesei. It is produced in an independent production line that can be set up onsite or offsite.

Using the alkaline pretreatment, the susceptibility to enzymatic hydrolysis is excellent, and the cellulose and hemicellulose polymers are converted into sugars called “very fermentable” (glucose, mannose), “poorly fermentable” (galactose), and “hardly fermentable” (xylose and arabinose). The enzymatic hydrolysis conditions, primarily the level of dry material of the mixture that is to be hydrolyzed and the amount of enzymes used, are selected in such a way that at the end of stage c), a conversion of between 20% and 90% of the cellulose of the pulp that circulates in the glucose pipe 9, and more particularly between 30% and 80%, is obtained.

The alcohol fermentation carried out in stage d) is ensured by yeasts or other microorganisms.

During stage e), the alcohols and/or solvents that are produced in stage d) are purified and separated.

Stage f) for separation of the cake can be carried out downstream from stages c), d) and/or e) and can optionally be coupled to a washing of the cake.

In all of the cases, at the outlet of stages c) to e) that are carried out in the reactor 10, a flow of products 11, optionally separated by any means that is known to one skilled in the art, a liquid residue 12 (called vinasse) containing unfermented sugars, and a solid cake 13 containing the solid material that is obtained from the initial substrate (solid residue), and a liquid fraction are obtained. The solid residue partly consists of cellulose that has not been hydrolyzed and that represents between 10% and 100% of this residue, and preferably between 30% and 70%.

The flow 13 that corresponds to the cake is divided into 3 fractions 13-1, 13-2 and 13-3.

The fraction 13-1 is sent to the top of the digester 2 with the initial vegetation 1, which corresponds to recycling upstream from the pretreatment stage a). It represents between 0 and 100% of the cake 13, and preferably between 20 and 100%.

The fraction 13-2 is sent downstream from the digester 6, and therefore upstream from the washing stage b) to be simply mixed under cold conditions with the paste 5. It represents between 0 and 95% of the cake 13, and preferably between 0 and 80%.

The fraction 13-3 that is not recycled is directly discharged beyond the process. It represents between 0 and 80% of the cake 13 and preferably less than 15%, and, even better, less than 10%.

### EXAMPLES

In all of the examples, dry material is denoted as ms.

FPu=Filter Paper Unit, which is a measurement of the enzymatic activity. The FPu−weight correspondence is a characteristic of the enzymatic cocktail.

### Example 1

### Material Balance—without Recycling (not in Accordance with the Invention)

A process for the production of ethanol from papermaking pulp obtained from a Kraft alkaline process is considered. The process treats 80 tons/hour of native vegetation. The vegetation is spruce (softwood), containing 55% by weight of dry material that consists of:

| | |
|---|---|
| Cellulose | 42% |
| Lignin | 30% |
| Hemicellulose | 15% |
| Others (Ashes, Extractibles . . .) | 13% |

The hemicelluloses consist of 50% mannans.

Kraft baking is carried out at 175° C. for 5 hours. This pretreatment and the washing processes carried out in stages a) and b) respectively are conducted in such a way that the papermaking pulp contains 15% dry material, and has preserved the following:

| | |
|---|---|
| Cellulose | 97% |
| Lignin | 10% |
| Hemicellulose | 52% |
| Others | 8% |

The ethanol conversion process consists of enzymatic hydrolysis of the papermaking pulp (stage c)), followed by alcohol fermentation into ethanol (stage d)), a separation of the solids in suspension for forming a solid residue or a cake, distillation, and then dehydration of ethanol at 99.7% by weight (stage e)).

The enzymatic hydrolysis is conducted under conditions such that the hydrolysis of 75% of cellulose and 55% of hemicelluloses is observed. 20 FPu/g of cellulose that enters into the hydrolysis reactor is consumed.

The fermentation makes it possible to transform 90% of the previously formed glucose and mannose into ethanol. The other sugars that are obtained from the hemicelluloses (xylose, arabinose, . . . ) are not fermented by the Saccharomyces cerevisae strain that is used.

Before the distillation stage, the solid residue is separated and washed to limit the loss of ethanol with the cake.

The conditions of the process are such that the exiting flows are:
Ethanol at 99.7% by weight: 6.96 tons/hour
Vinasse: 143.64 tons/hour
Solid cake: 22.96 tons/hour with 36% solid material. The solid part is 54.1% of the non-hydrolyzed cellulose.

The ethanol yield of this process is therefore 15.8% by weight on the native vegetation (dry base material). The specific enzyme consumption is 51,540 FPu/kg of ethanol that is produced.

The so-called “recalcitrant” cellulose that is present in the cake has a hydrolysis yield (under the conditions of the process above) and with the same enzymatic feedstock that will be only 30%.

### Example 2

### Material Balance—with Recycling of the Solid Residue at the Level of the Enzymatic Hydrolysis (not in Accordance with the Invention)

It is possible to recycle the cake at the enzymatic hydrolysis stage so as to limit the losses of cellulose. Nevertheless, in the absence of treatment, the so-called “recalcitrant” cellulose has very reduced sensitivity to enzymatic hydrolysis relative to the cellulose of the papermaking paste. Its hydrolysis yield (under the conditions of the process above), and with the same enzymatic feedstock, will be only 30%. In addition, the recycling brings about the accumulation of insoluble products (lignin) in the process, and the fermentation, under the conditions of the process above, is to be carried out with a maximum of 8% of solid material in the reaction medium. Thus, it is necessary to limit the amount of recycled cake, and, in practice, only 68% of the cake can be recycled. The following exiting flows of the process are then obtained:
Ethanol at 99.7% by weight: 7.92 tons/hour, or 14% more than Example 1.
Vinasse: 198.06 tons/hour
Solid cake: 17.14 tons/hour with 36% solid material (non-recycled part). The solid part is 50.3% of the non-hydrolyzed cellulose.

The ethanol yield of this process is therefore 18.0% by weight on the native vegetation (dry base material) or an improvement of 2.2 points relative to the basic case. Nevertheless, this improvement of the mass balance is achieved to the detriment of the specific enzyme consumption that is then 61,740 FPu/kg of ethanol produced (+20%) and requires a larger reaction volume: +56% for enzymatic hydrolysis, or an increase of the specific volume (relative to the production) of 36%.

Thus, the improvement of the mass balance makes it possible to reduce the contribution of the cost of the raw material in the final production cost of ethanol, but the expense items “enzymes” and “investments” are increased significantly.

### Example 3

### Material Balance—with Recycling of the Solid Residue at the Top of the Digester (According to the Invention)

On the basis of the process that is described in Example 1, a recycling of 90% of the solid residue that is created is introduced at the inlet of the digester 2 (upstream from the pretreatment stage a)).

Thus, 24.40 tons/hour of moist cake that contains 55.7% of cellulose are recycled to the digester to make it undergo this treatment in the same unit as the native vegetation. Because of the absence of ligneous protection on the fibers of the cake, the alkaline Kraft treatment will produce more significant losses of materials than on the native vegetation. The recycled paste thus preserves the following at the outlet of stages a) and b):

| | |
|---|---|
| Cellulose | 80% |
| Lignin | 10% |
| Hemicellulose | 35% |
| Others | 10% |

The hydrolysis and fermentation conditions are preserved. The hydrolysis of the native vegetation has the same yield. Because of the significant swelling of the cellulose fibers that are recycled in an alkaline medium and in the absence of lignin surrounding these fibers upon their input into the digester for the chemical alkaline pretreatment, the cellulose recovers all of its susceptibility to enzymatic hydrolysis and therefore has a hydrolysis yield that is equal to that of the cellulose that is obtained from the native vegetation (75%). The hemicelluloses also recover a yield of 55%. The conditions of alcohol fermentation and separation are preserved. Thus, owing to the process according to the invention, exiting flows of the process are obtained:
Ethanol at 99.7% by weight: 8.41 tons/hour, or 21% more than Example 1.
Vinasse: 167.17 tons/hour
Solid cake: 2.71 tons/hour (non-recycled part), containing 36% solids. The solid part is 55.7% of the cellulose.

The ethanol yield of this process is therefore 19.1% by weight on the native vegetation (dry base material) or 3.3 points more than Example 1 and 1.1 points more than Example 2. Furthermore, the specific enzyme consumption has increased only very slightly and is 51,870 FPu/kg of ethanol that is produced, or an only 0.6% increase. The reaction volume that is involved is 20% more than Example 1, and therefore the specific volume is the same as for Example 1. Furthermore, 8.7 tons of solids are sent to the Kraft treatment in addition to 44 tons of solids of native vegetation, or an increase of 20%, proportional to the increase in production.

The implementation of the process according to the invention makes it possible to greatly improve the mass balance and therefore to decrease the contribution of the cost of the raw material in the final production cost of ethanol. The recycling upstream from the pretreatment stage makes it possible to monitor the lignin level in the process and therefore makes it possible to recycle a larger amount than Example 2, while preserving a correct level of solids in fermentation, which leads to an even better material yield. In addition, the invention makes it possible to preserve the contribution of expense items “enzymes” and “investments” in the case without recycling. Nevertheless, an increase in expense items of the pretreatment will be noted because of the increase of the material that is to be treated.

### Example 4

### Material Balance—with Recycling Partially Upstream from the Digester 2 and Partially Upstream from the Washing Reactor 6 (According to the Invention)

On the basis of the process that is described in Example 1, a recycling of 25% of the cake that is created is introduced upstream from the digester 2, and 70% is introduced into the washing reactor 6, i.e., downstream from the digester 2. The recycling cost at the washing is lower than the recycling at the digester.

Thus, 11.06 tons/hour of moist cake that contains 46.2% of the cellulose are recycled at the digester (at the top of the Kraft pretreatment), and 30.95 tons/hour are recycled in the washing reactor. The hydrolysis and fermentation conditions are preserved. The hydrolysis of the native vegetation has the same yield. Because of the significant swelling of the cellulose fibers that are recycled in the alkaline medium—and in the absence of lignin surrounding these fibers upon their input into the digester for the chemical alkaline pretreatment—the cellulose recovers all of its susceptibility to enzymatic hydrolysis and therefore has a hydrolysis yield that is equal to that of the cellulose that is obtained from the native vegetation (75%). The hemicelluloses also recover a yield of 55%. The losses that are due to the Kraft pretreatment are the same as in Example 3. The cellulose that is recycled at the inlet of the washing reactor is also subjected to an alkaline medium, but with lower basicity and at more moderate temperatures (40° C.); the swelling of the fibers is thus more limited than during Kraft digestion, but nevertheless present, which makes it possible to obtain an increase in the hydrolysis yield of two-thirds relative to the recalcitrant cellulose of the cake (or 50%). The washing makes it possible to preserve 99% of the solids of the cake and a portion of the soluble products that are present in the liquid portion of the cake (50%). The alcohol fermentation conditions are preserved. Thus, owing to the installation of the invention, the following exiting flows of the process are obtained:
Ethanol at 99.7% by weight; 8.93 tons/hour
Vinasse: 199.41 tons/hour
Solid cake: 2.21 tons/hour (non-recycled part).

The ethanol yield of this process is therefore 20.3% by weight on the native vegetation (dry base material) or 4.5 points more than Example 1 and 2.3 points more than Example 2. The specific enzyme consumption has increased to 54,750 FPu/kg of ethanol that is produced, or a 6.2% increase relative to Example 1, but is still much less than Example 2. The reaction volume involved is 52% more than Example 1, which corresponds to an increase of the specific volume of 18.4% relative to Example 1 and is still much less than Example 2. Relative to the pretreatment, only 3.9 tons of solids are sent to the Kraft treatment in addition to 44 tons of solids of native vegetation, or an increase of 9%, which is much lower than the increase in production.

The implementation of the process according to the variant of the invention has made it possible to greatly improve the mass balance and therefore to decrease the contribution of the cost of the raw material in the final production cost of ethanol. The recycling according to the invention makes it possible to monitor the lignin level in the process and therefore makes it possible to recycle a larger amount than Example 2, while preserving a correct level of solids in fermentation, which leads to an even better material yield. Relative to Example 3, this variant of the process of the invention makes it possible to improve the material balance while limiting the amount of solids sent to the Kraft pretreatment and therefore represents a lower retreatment cost than the one mentioned in Example 3 where the recycling is done entirely at the pretreatment stage. Nevertheless, in contrast, the items “enzymes” and “investments” are increased.

The economic optimum of an installation depends on the relative cost of the expense items and primarily the cost of the raw material, the pretreatment, investments, and enzymes that are used. This embodiment of the invention makes it possible to greatly improve the material balance of the process, with a limited—and even zero—impact on the other items. According to the economic data of an installation, this embodiment will be used to greatly improve the profitability of the process.

### Example 5

### Material Balance—without Recycling (not in Accordance with the Invention)

A process for the production of an acetone-butanol-ethanol (ABE) mixture starting from papermaking pulp obtained from a Kraft alkaline process is considered. The process treats 150 tons/hour of native vegetation. The vegetation is eucalyptus (hardwood), containing 50% by weight of dry material that consists of:

| | |
|---|---|
| Cellulose | 45% |
| Lignin | 22% |
| Hemicellulose | 17% |
| Others | 16% |

The hemicelluloses consist of C5 sugars (xylans and arabinans).

Kraft baking is carried out at 165° C. for 2.5 hours. This pretreatment and the washing processes carried out in stages a) and b) respectively are conducted in such a way that the papermaking pulp contains 10% dry material, and has preserved the following:

| | |
|---|---|
| Cellulose | 98.5% |
| Lignin | 9% |
| Hemicellulose | 65% |
| Others | 20% |

The ethanol conversion process consists of enzymatic hydrolysis of the papermaking pulp (stage c), a separation of solids in suspension for forming a cake with washing for maximizing the recovery of sugars, and then an ABE fermentation of the liquid phase that contains the sugars (stage d)), and the distillation of the ABE (stage e). It should be noted that the ABE fermentation uses the sugars both with 6 atoms and with 5 atoms of carbon (glucose and xylose).

The enzymatic hydrolysis is conducted under conditions such that the hydrolysis of 85% of the cellulose and 65% of the hemicelluloses is observed. 25 FPu/g of cellulose entering the hydrolysis reactor is consumed.

Before the fermentation stage, the solid residue is separated and washed for limiting the loss of sugar with the cake.

The fermentation makes it possible to transform the glucose and the xylose previously formed into an ABE mixture, producing 0.3 g of ABE per g of sugar present.

The conditions of the process are such that the exiting flows are:
ABE (pure): 11.16 tons/hour
Vinasse: 416.95 tons/hour
Solid cake: 33.74 tons/hour with 33.3% solid material. The solid part is 44.3% of the non-hydrolyzed cellulose.

The ABE yield of this process is therefore 14.9% by weight on the native vegetation (dry material base). The specific enzyme consumption is 74,470 FPu/kg of ABE that is produced.

The so-called “recalcitrant” cellulose that is present in the cake has a hydrolysis yield (under the conditions of the process above) and with the same enzymatic feedstock that will be only 25%.

### Example 6

### Material Balance—with Recycling of the Solid Residue at the Level of Enzymatic Hydrolysis (not in Accordance with the Invention)

It is possible to recycle the cake at the enzymatic hydrolysis stage so as to limit the losses of cellulose. Nevertheless, in the absence of treatment, the so-called “recalcitrant” cellulose has very reduced sensitivity to enzymatic hydrolysis relative to the cellulose of the papermaking paste. Its hydrolysis yield (under the conditions of the process above), and with the same enzymatic feedstock, will be only 25%. In addition, the recycling brings about the accumulation of insoluble products (lignin) in the process, which leads to larger volumes of hydrolysis reactors. 90% of the thus formed cake is recycled. The following exiting flows of the process are then obtained:
ABE (pure): 12.84 tons/hour, or 15% more than Example 5.
Vinasse: 787.41 tons/hour
Solid cake: 17.10 tons/hour with 33.3% solid material (non-recycled part). The solid part is 26.9% of the non-hydrolyzed cellulose.

The ABE yield of this process is therefore 17.1% by weight on the native vegetation (dry material base) or an improvement of 2.2 points relative to the basic case. Nevertheless, this improvement of the mass balance is achieved to the detriment of the specific enzyme consumption that is then 91,590 FPu/kg of ABE produced (+23%) and requires a reaction volume that has more than doubled: +113% for enzymatic hydrolysis or an increase of the specific volume (relative to the production) by +85%.

Thus, the improvement of the mass balance makes it possible to reduce the contribution of the cost of the raw material in the final production cost of ABE, but the expense items “enzymes” and primarily “investments” are increased significantly.

### Example 7

### Material Balance—with Recycling of the Solid Residue at the Top of the Digester (According to the Invention)

On the basis of the process that is described in Example 5, a recycling of 90% of the cake (solid residue) that is created is introduced into the inlet of the digester 2 (upstream from the Kraft pretreatment stage).

Thus, 34.77 tons/hour of moist cake that contains 43.8% of cellulose are recycled at the Kraft top to make it undergo this treatment in the same unit as the native vegetation. Because of the absence of ligneous protection on the fibers of the cake, the Kraft treatment will produce more significant losses of materials than on the native vegetation. The recycled paste thus preserves the following at the outlet of stages a) and b):

| | |
|---|---|
| Cellulose | 86% |
| Lignin | 9% |
| Hemicellulose | 54% |
| Others | 15% |

The hydrolysis and fermentation conditions are preserved. The hydrolysis of the native vegetation has the same yield. Because of the significant swelling of the cellulose fibers that are recycled in an alkaline medium and in the absence of lignin surrounding these fibers upon their input into the digester for the chemical alkaline pretreatment, the cellulose recovers all of its susceptibility to enzymatic hydrolysis and therefore has a hydrolysis yield that is equal to that of the cellulose that is obtained from the native vegetation (85%). The hemicelluloses also recover a yield of 65%. The conditions of ABE fermentation and separation are preserved. Thus, owing to the process according to the invention, exiting flows of the process are obtained:
ABE (pure): 12.73 tons/hour, or 14.1% more than Example 5.
Vinasse: 472.15 tons/hour
Solid cake: 3.86 tons/hour (non-recycled part), containing 33.3% solids. The solid part is 43.8% of the cellulose.

The ABE yield of this process is therefore 17.0% by weight on the native vegetation (dry base material), or 2.1 points more than Example 5 and 0.1 point less than Example 6. Furthermore, the specific enzyme consumption has decreased only very slightly and is 73,835 FPu/kg of ABE that is produced, or 0.8% reduction. The reaction volume that is involved is 14.3% more than Example 5, and therefore the specific volume is the same as for Example 5. Furthermore, 11.6 tons of solids are sent to the Kraft treatment in addition to 75 tons of solids of native vegetation, or an increase of 15.5%, slightly more than the increase in production.

The implementation of the process according to the invention made it possible to greatly improve the mass balance and therefore to decrease the contribution of the cost of the raw material in the final production cost of ABE. The recycling according to the invention makes it possible to monitor the lignin level in the process and therefore makes it possible to limit the volume that is necessary to the hydrolysis relative to Example 6.

### Example 8

### Material Balance—with Recycling Partially Upstream from the Digester 2 and Partially Upstream from the Washing Reactor 6 (According to the Invention)

On the basis of the process that is described in Example 5, a recycling of 30% of the cake that is created is introduced upstream from the digester 2, and 60% is introduced into the washing reactor 6, i.e., downstream from the digester 2.

Thus, 19.53 tons/hour of moist cake that contains 36.4% of cellulose are recycled at the digester (upstream from the Kraft pretreatment), and 39.06 tons/hour are recycled in the washing reactor. The hydrolysis and fermentation conditions are preserved. The hydrolysis of the native vegetation has the same yield. The cellulose recovers all of its susceptibility to enzymatic hydrolysis and therefore has a hydrolysis yield that is equal to that of the cellulose that is obtained from the native vegetation (85%). The hemicelluloses also recover a yield of 65%. The losses that are due to the Kraft pretreatment are the same as in Example 7. The cellulose that is recycled at the inlet of the washing reactor is also subjected to an alkaline medium, but with lower basicity and at much more moderate temperatures (40° C.); the swelling of the fibers is thus more limited than during Kraft digestion, but nevertheless present, which makes it possible to obtain an increase in the hydrolysis yield of 80% relative to the recalcitrant cellulose of the cake (or 45%). The washing makes it possible to preserve 99% of the solids of the cake and a portion of the soluble products that are present in the liquid portion of the cake (50%). The ABE fermentation conditions are preserved. Thus, owing to the installation of the invention, the following exiting flows of the process are obtained:
ABE (pure): 12.84 tons/hour, or 15% more than Example 5
Vinasse: 542.68 tons/hour
Solid cake: 6.51 tons/hour (non-recycled part), containing 33.3% solids. The solid part is 36.4% of the cellulose.

The ABE yield of this process is therefore 17.1% by weight on the native vegetation (dry base material) or 2.2 points more than Example 5 and the same as Example 6. The specific enzyme consumption has increased to 77,940 FPu/kg of ABE that is produced, or a 4.7% increase relative to Example 5, but is still much less than Example 6. The reaction volume involved is 35.7% more than Example 5, which corresponds to an increase of the specific volume of 18% relative to Example 5 and is still much less than Example 6. Relative to the pretreatment, only 6.5 tons of solids are sent to the Kraft treatment in addition to 75 tons of solids of native vegetation, or an increase of 8.7%, which is much lower than the increase in production.

The implementation of the process according to this variant has made it possible to greatly improve the mass balance and therefore to decrease the contribution of the cost of the raw material in the final production cost of ABE. The recycling according to the invention makes it possible to monitor the lignin level in the process and therefore makes it possible to limit the volume that is necessary to the hydrolysis relative to Example 6. Relative to Example 7, this embodiment makes it possible to improve the material balance while limiting the amount of solids sent to the Kraft pretreatment and therefore makes it possible to decrease the retreatment cost. Nevertheless, in contrast, the items “enzymes” and “investments” are increased, but are still lower than in the case of a “direct” recycling.

1-14. (canceled)

15. A process for the production of alcohols and/or solvents from cellulosic or lignocellulosic biomass that comprises at least the following stages:
a) Alkaline chemical pretreatment based on sodium sulfate of a cellulosic or lignocellulosic substrate;
b) Washing of the pretreated substrate;
c) Enzymatic hydrolysis of the substrate that is pretreated and washed using cellulolytic and/or hemicellulolytic enzymes that produce a hydrolyzate and a water-insoluble residue;
d) Microorganism fermentation of the hydrolyzate that is obtained from stage c) and production of a fermentation must that contains at least one alcohol and/or solvent;
e) Separation/purification of alcohol and/or solvent, and
f) Separation of a cake that contains the water-insoluble residue,
in which at least a portion of the cake that is obtained in stage f) is recycled upstream from the pretreatment stage a) and also upstream from the washing stage b).

16. The production process according to claim 15, in which at least one fraction that represents between 0 and 100% of the cake is sent upstream from the pretreatment stage a).

17. The process according to claim 16, in which the fraction represents between 20 and 100% of the cake.

18. The process according to claim 15, in which at least one fraction that represents up to 95% of the cake is sent upstream from the washing stage b).

19. The process Process according to claim 18, in which the fraction represents up to 80% of the cake.

20. The process according to claim 15, in which at least one fraction that represents up to 80% of the cake is directly discharged without recycling.

21. The process according to claim 20, in which the fraction represents less than 15% of the cake.

22. The process according to claim 15, in which the pretreatment stage a) is carried out at a temperature of between 150 and 180° C.

23. The process according to claim 15, in which stage c) for enzymatic hydrolysis is carried out by means cellulases and/or hemicellulases that are produced by a microorganism that is a fungus that belongs to the genera Trichoderma, Aspergillus, Penicillium or Schizophyllum, or an anaerobic bacteria that belongs to the genus Clostridium.

24. The process according to claim 15, in which stages c) and d) are coupled in the same reactor.

25. The process according to claim 15, in which stage c) is carried out in such a way that between 20 and 90%—of the cellulose that is contained in the pretreated and washed substrate is converted into glucose.

26. The process according to claim 15, in which the alcohol that is obtained at the end of stage e) is ethanol.

27. The process according to claim 15, in which the solvent that is obtained at the end of stage e) is an acetone-butanol-ethanol mixture.

28. The process according to claim 16, in which stage f) for separation of the cake is carried out downstream from stages c), d) and/or e) and optionally is coupled to a washing of the cake.
